(19) United States
(12) Patent Application Publication
(10) Pub. No.: US 2014/0343682 A1
(43) Pub. Date: Nov. 20, 2014

(54) CONVERTIBLE ACETABULAR BEARING

(71) Applicant: Zimmer, Inc., Warsaw, IN (US)

(72) Inventor: Greg Mangan, Warsaw, IN (US)

(21) Appl. No.: 14/279,940

(22) Filed: May 16, 2014

Related U.S. Application Data
(60) Provisional application No. 61/824,564, filed on May 17, 2013.

(51) Int. Cl. A61F 2/34

(52) U.S. Cl. CPC A61F 2/34; A61F 2002/3446
USPC 623/22.24; 623/22.32

(57) ABSTRACT

An acetabular bearing is configured to be convertible between direct connection with the acetabulum of a patient's hip, and connection to an acetabular cup shell or other mounting structure, which is configured to be connected to the acetabulum.

### CLAIM OF PRIORITY

This application claims the benefit of U.S. Provisional Patent Application Ser. No. 61/824,564, filed on May 17, 2013, the benefit of priority of which is claimed hereby, and which is incorporated by reference herein in its entirety.

### BACKGROUND

Total hip replacement surgery is commonly performed to alleviate pain and loss of function in injured and diseased hip joints. During this surgery, the articulating surfaces of the hip joint are replaced with prosthetic bearing components. The replacement components generally include a femoral component having a convex bearing surface and an acetabular cup component having a mating concave bearing surface. The femoral bearing is configured to rotate in the acetabular bearing in a manner that approximates the rotation of a patient's femoral head in the acetabulum of the hip.

Modular femoral and acetabular components are useful, at least in part, because they allow the surgeon to assemble components in a variety of configurations at the time of surgery to meet specific patient needs relative to size and geometry. For example, modular femoral components generally include separate stem and head components that can be assembled in a variety of configurations of surface finish, stem diameter, stem length, proximal stem geometry, head diameter, and neck length. Likewise, modular acetabular components generally include separate shell and liner components that can be assembled in a variety of configurations of surface finish, shell outer diameter, liner inner diameter, and constraining fit with the femoral head.

### SUMMARY

Examples according to this disclosure include an acetabular bearing that is configured to be convertible between direct connection with the acetabulum of a patient's hip, and connection to an acetabular cup shell or other mounting structure, which is configured to be connected to the acetabulum.

In one example, an acetabular bearing includes a radially inner concave hemispherical surface that is configured to receive a portion of at least one of a femur or a femoral prosthesis, and a radially outer convex hemispherical surface including at least one surface feature that configures the acetabular bearing to be affixed to an acetabulum and at least one surface feature that configures the acetabular bearing to be coupled to a mounting structure that is configured to be affixed to an acetabulum.

In another example, a prosthesis includes an acetabular cup shell and an acetabular bearing. The acetabular cup shell includes a first radially inner hemispherical concave surface, and a first radially outer convex hemispherical surface configured to be received in and affixed to an acetabulum. The acetabular bearing includes a second radially inner concave hemispherical surface that is configured to receive a portion of at least one of a femur or a femoral prosthesis, and a second radially outer convex hemispherical surface configured to be received in the first radially inner concave hemispherical surface of the acetabular cup shell. The second radially outer convex hemispherical surface includes at least one surface feature that configures the acetabular bearing to be affixed to an acetabulum and at least one surface feature that configures the acetabular bearing to be coupled to the acetabular cup shell.

In another example, a method includes providing an acetabular bearing and affixing the acetabular bearing to the acetabulum. The acetabular bearing includes a radially inner concave hemispherical surface that is configured to receive a portion of at least one of a femur or a femoral prosthesis, and a radially outer convex hemispherical surface including a first surface feature that configures the acetabular bearing to be affixed to an acetabulum and a second surface feature that configures the acetabular bearing to be coupled to a mounting structure that is configured to be affixed to the acetabulum. The acetabular bearing is affixed to the acetabulum via the first surface feature.

In another example, a method includes providing an acetabular cup shell, providing an acetabular bearing, affixing the acetabular cup shell to the acetabulum, and coupling the acetabular bearing to the acetabular cup shell. The acetabular cup shell includes a first radially inner hemispherical concave surface, and a first radially outer convex hemispherical surface configured to be received in and affixed to an acetabulum. The acetabular bearing includes a second radially inner concave hemispherical surface that is configured to receive a portion of at least one of a femur or a femoral prosthesis, and a second radially outer convex hemispherical surface configured to be received in the first radially inner concave hemispherical surface of the acetabular cup shell. The second radially outer convex hemispherical surface includes a first surface feature that configures the acetabular bearing to be affixed to the acetabulum and a second surface feature that configures the acetabular bearing to be coupled to the acetabular cup shell. The acetabular bearing is coupled to the acetabular cup shell via the second surface feature.

The details of examples of the disclosure are set forth in the accompanying drawings and the description below. Other features, objects, and advantages of examples according to this disclosure will be apparent from the description and drawings, and from the claims.

### BRIEF DESCRIPTION OF DRAWINGS

FIGS. 1A and 1B are section views of two example hip prosthesis including a convertible acetabular bearing in accordance with this disclosure.

FIGS. 2A and 2B are a plan view and a section view, respectively, of an example convertible acetabular bearing.

FIG. 3 is a section view of an example convertible acetabular bearing coupled to an acetabular cup shell.

FIGS. 4A-4C are broken section views of a portion of acetabular bearings in accordance with this disclosure.

FIG. 5 is a flowchart illustrating a method of implanting a prosthesis including a convertible acetabular bearing in accordance with this disclosure.

FIG. 6 is a flowchart illustrating another method of implanting a prosthesis including a convertible acetabular bearing in accordance with this disclosure.

### DETAILED DESCRIPTION

As noted above, modular hip prosthesis components are useful, at least in part, because they allow the surgeon to assemble components in a variety of configurations at the time of surgery to meet specific patient needs relative to size and geometry. Hip prostheses commonly include an acetabular component and a femoral component. The acetabular component is configured to be received by and attached to the acetabulum of a patient's hip. The acetabular component of a hip prosthesis provides a bearing surface that replaces the bearing surface previously provided by the acetabulum of the patient's hip.

Acetabular components come in a variety of configurations. In some cases, acetabular components include an acetabular cup shell, which is configured to be connected to the acetabulum of a patient's hip, and a liner, which is configured to be connected to the shell and provide the bearing surface that replaces the acetabulum of the patient's hip. In other cases, a single acetabular cup bearing is connected directly to the patient's hip and provides the bearing surface that replaces the acetabulum of the patient's hip. The use of an acetabular bearing with or without an intermediate shell may be based on physician preference, patient anatomy, or other factors. In any event, the acetabular bearing that is configured to be connected directly to the acetabulum is designed with different features than the acetabular bearing that is configured to be connected to the shell, as the requirements of securely connecting the bearing to the acetabulum are different than connecting the bearing to an intermediate acetabular cup shell.

Examples according to this disclosure include an acetabular bearing that is configured to be convertible between direct connection with the acetabulum of a patient's hip, and connection to an acetabular cup shell, which shell is configured to be connected to the acetabulum. Reference to “direct” connection of an acetabular bearing to an acetabulum of a patient's hip in this disclosure refers to connecting the bearing to the acetabulum without any additional prosthetic components interposed between the bearing and the acetabulum. However, such a direct connection may be achieved via a layer of adhesive interposed between the outer surface of the bearing and the acetabulum.

Acetabular bearings in accordance with this disclosure provide improved modularity to hip prostheses, which may improve physician satisfaction and improve surgical outcomes. Additionally, manufacturing and other costs associated with producing and selling hip prostheses can be decreased by providing a single acetabular bearing that can be employed in a variety of different modalities.

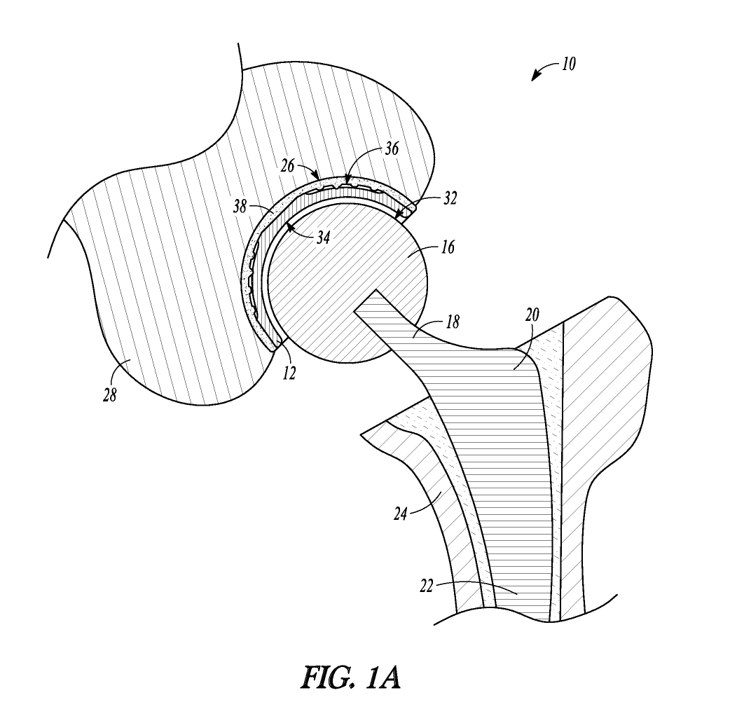

FIGS. 1A and 1B depict an example of a hip prosthesis 10 including an acetabular bearing 12 in accordance with this disclosure. Acetabular bearing 12 is configured to be connected, whether directly or indirectly, to acetabulum 26 of a patient's hip 28. In FIGS. 1A and 1B, example hip prosthesis 10 also includes femoral prosthesis 14. Femoral prosthesis 14 includes femoral head 16, neck 18, body 20, and stem 22. Femoral prosthesis 14 is configured to be implanted within and replace a portion of femur 24 of a patient. Femoral head 16 is configured to act as a bearing that is received by and rotates in acetabular bearing 12 in a manner that approximates the rotation of the patient's femoral head in acetabulum 26 of hip 28. As described in more detail below, example acetabular bearing 12 is configured to be convertible between direct connection with acetabulum 26 of hip 28, as illustrated in FIG. 1A, and connection to acetabular cup shell 30, which shell 30 is then connected to acetabulum 26 of hip 28, as illustrated in FIG. 1B.

In some examples, one or more of femoral head 16, neck 18, body 20, and stem 22 of femoral prosthesis 14 are separate components that are mechanically connected to one another. In some examples, femoral component 14 includes a single integral structure, different portions of which include head 16, neck 18, body 20, and stem 22. In one example, neck 18, body 20, and stem 22 of femoral component 14 are fabricated as a single integral component to which a separate femoral head 16 is connected.

As illustrated in FIGS. 1A and 1B, femoral prosthesis 14 is received by and affixed to femur 24. For example, body 20 and stem 22 are placed in a receptacle formed in femur 24. In some examples, stem 22 is attached to femur 24 using bone cement or another appropriate fixation system. Bone cements and other adhesives are described below with reference to acetabular bearing 12 and such adhesives can also be used to affix femoral prosthesis 14 to femur 24.

Head 16 of femoral prosthesis 16 includes a generally spherical convex bearing surface 32, which is configured to be received in a generally hemispherical concave bearing surface 34 of acetabular bearing 12. Whether integral or separate components mechanically coupled, femoral head 16 and neck 18, body 20, and stem 22 of femoral prosthesis 14 are joined such that movement of femur 24 causes all of the portions of femoral prosthesis to move together. Notably, femoral head 16 and neck 18 are coupled such that the components can neither rotate nor translate relative to one another.

Femoral prosthesis 14 including femoral head 16, neck 18, body 20, and stem 22 can be fabricated from a variety of biologically compatible materials and by a variety of processes including machining, casting, forging, compression molding, injection molding, sintering, and/or other suitable processes. In some examples, all of the portions of femoral prosthesis 14 are fabricated from the same material, while, in other examples, different portions of femoral prosthesis 14 are fabricated from different materials. In one example, one or more portions of femoral prosthesis 14 are fabricated from metals, polymers, ceramics, and/or other suitable materials. For example, one or more portions of femoral prosthesis 14 may be made of a cobalt-chromium alloy. Other metals suitable for femoral prosthesis 14 (including in combination with cobalt and/or chrome) include titanium, aluminum, vanadium, molybdenum, hafnium, nitinol, molybdenum, tungsten, nickel, tantalum, and stainless steel.

Example acetabular bearing 12 includes a generally hemispherical concave bearing surface 34, which is configured to receive the spherical convex bearing surface 32 of femoral head 16 of femoral prosthesis 14. Acetabular bearing 12 also includes a radially outward, generally hemispherical convex surface 36, by which acetabular bearing 12 is coupled either directly to acetabulum 26 of hip 28 or to acetabular cup shell 30.

In FIG. 1A, hip prosthesis includes acetabular bearing 12 directly connected to acetabulum 26 of hip 28. In other words, acetabular bearing 12 is coupled to the patient's hip without any intermediate prosthetic devices or structures like acetabular cup shell 30 or another mounting device. Acetabular bearing 12 is mechanically coupled to acetabulum 26, at least in part, by a layer of adhesive, which, in the example of FIG. 1A, includes a mantel 38 of bone cement.

A variety of materials configured for adequately stabilizing a prosthetic component to a bone can be used to adhere acetabular bearing 12 directly to acetabulum 26, as illustrated in the example of FIG. 1A. For example, cement mantel 38 may include a polymeric bone cement, such as a polymethyl methacrylate (“PMMA”) cement. In some examples, cement mantel 38 includes a powder capable of being mixed with a liquid, or a liquid or gel which hardens into a solid material.

In some examples, cement mantel 38 is formed from a cement precursor including one or more materials that undergo polymerization or cross-linking to form a solid or substantially solid mantel 38, when cured. The cement precursor can be formed by mixing a liquid monomer with a particulate or powdered copolymer. After the liquid monomer and the copolymer are mixed and applied, the liquid monomer can undergo a polymerization reaction, such as a free-radical polymerization, to form the solid or substantially solid cement mantel 38.

In some examples, a liquid methyl methacrylate monomer can be used in the formation of cement mantel 38. Additionally, a particulate or powdered methyl methacrylate-styrene copolymer can be used in cement mantel 38. Other compounds, such as a polymerization initiator or a polymerization accelerator, can be mixed with a liquid monomer and a particulate or powdered copolymer when forming the cement precursor that undergoes a polymerization reaction to form the solid or substantially solid cement mantel 38. Cement mantel 38 can also include other moldable materials, such as biodegradable polymers, for example, polyhydroxyalkanoate. An example of a suitable bone cement that can be used to form cement mantel 38 is OSTEOBOND® copolymer bone cement, manufactured by Zimmer, Inc., of Warsaw, Ind.

In the example of FIG. 1B, hip prosthesis 10 includes acetabular bearing 12 connected to acetabular cup shell 30, which is connected to acetabulum 26 of hip 28. Acetabular cup shell 30 includes a generally hemispherical body having a convex radially outward surface 40 that is configured to be received in the concave surface of acetabulum 26. Radially inward surface 42 of shell 30 is a concave hemispherical surface that is configured to receive outward convex surface 36 of acetabular bearing 12. Acetabular cup shell 30 can be mechanically coupled to acetabulum 26 in a variety of ways. In the example of FIG. 1B, acetabular cup shell 30 is mechanically coupled to acetabulum 26, at least in part, by a layer of adhesive, which, in the example of FIG. 1B, includes a mantel 44 of bone cement.

A variety of materials configured for adequately stabilizing a prosthetic component to a bone can be used to adhere acetabular cup shell 30 to acetabulum 26. For example, cement mantel 44 may include a polymeric bone cement, such as a polymethyl methacrylate (“PMMA”) cement. In some examples, cement mantel 44 includes a powder capable of being mixed with a liquid, or a liquid or gel which hardens into a solid material. Additionally, the other adhesives, precursors, and other materials and processes described above with reference to cement mantel 38 can also be employed in the formation of cement mantel 44 to affix acetabular cup shell 30 to acetabulum 26 of hip 28.

Acetabular cup shell 30 can be fabricated by a variety of processes including machining, casting, forging, compression molding, injection molding, sintering, and/or other suitable processes. Additionally, acetabular cup shell 30 can be fabricated from a variety of materials including metals, polymers, ceramics, and/or other suitable materials. For example, shell 30 can be fabricated from a cobalt-chromium alloy.

Acetabular bearing 12 can also be fabricated by a variety of processes including machining, casting, forging, compression molding, injection molding, sintering, and/or other suitable processes. Additionally, acetabular bearing 12 can be fabricated from a variety of materials including metals, polymers, ceramics, and/or other suitable materials. For example, acetabular bearing 12 can be fabricated from a cobalt-chromium alloy. In one example, acetabular bearing 12 is fabricated from polyethylene. In another example, acetabular bearing 12 is fabricated from a ceramic, including, e.g., a zirconia or zirconia, alumina ceramic. In one example, acetabular bearing 12 is fabricated from BIOLOX® ceramics manufactured by CeramTec BmbH of Lauf, Germany, including BIOLOX® detla or BIOLOX® forte.

In the example of FIG. 1B, in which hip prosthesis 10 includes acetabular bearing 12 connected to acetabular cup shell 30, bearing 12 and shell 30 may be fabricated from different materials. For example, acetabular bearing 12 may be made of polyethylene and acetabular cup shell 30 from a metal such as a cobalt-chromium alloy. In this example, because of the differing expansion coefficients of metal and polyethylene, acetabular bearing 12 will expand more than acetabular cup shell 30 when the assembly is warmed by the patient's body. This differential expansion can act to further lock acetabular bearing 12 to shell 30 to resist relative motion between them.

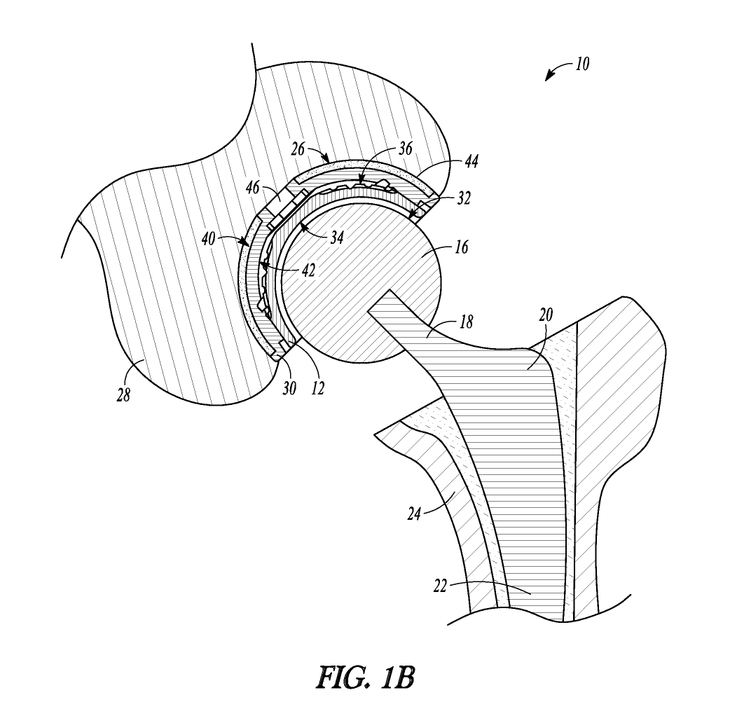

As illustrated in the examples of FIGS. 1A and 1B and described above, acetabular bearing 12 is configured to be convertible between direct connection with acetabulum 26 of hip 28, as illustrated in FIG. 1A, and connection to acetabular cup shell 30, which shell 30 is then connected to acetabulum 26 of hip 28, as illustrated in FIG. 1B. In particular, acetabular bearing 12 includes a number of structural features on radially outward convex surface 36, which configure acetabular bearing 12 to alternatively connect directly to acetabulum 26 or connect to acetabular cup shell 30, which shell 30 is then connected to acetabulum 26. In one example, convex surface 36 of acetabular bearing 12 includes at least on surface feature that configures bearing 12 to be mechanically coupled to acetabulum and at least one surface feature that configures bearing 12 to be mechanically coupled to acetabular cup shell 30. Different examples of the manner in which acetabular bearing 12, and other such acetabular bearings in accordance with this disclosure are configured to be alternatively connected directly to an acetabulum and an intermediate mounting structure such as an acetabular cup shell is described in greater detail below with reference to FIGS. 2A-4B.

In use, an acetabular prosthesis, whether including just acetabular bearing 12 or bearing 12 and shell 30, lines acetabulum 26 on the pelvic side of hip 28. Acetabular bearing 12 or acetabular bearing 12 connected to acetabular cup shell 30 is pressed into the prepared acetabulum 26 of hip 28. In the example of FIG. 1A, acetabular bearing 12 is affixed to acetabulum 26 by cement mantel 38 to lock bearing 12 in place. In the example of FIG. 1B, acetabular cup shell 30 is similarly affixed to acetabulum 26 by cement mantel 44 to lock shell 30 in place. However, in other examples, acetabular cup shell 30 may abut acetabulum and be affixed thereto by other appropriate fixation devices, including, e.g., one or more bone screws. Additionally, in the example of FIG. 1B, in addition to cement mantel 44, acetabular cup shell 30 may also be secured to acetabulum 26 by one or more bone screws, e.g., a bone screw through hole 46 generally aligned with the pole of hemispherical shell 30.

Femoral prosthesis 14 replaces the natural femoral head of a patient. Stem 22 of femoral prosthesis 14 is seated in a prepared intramedullary space of the femur 24. Femoral stem 22 may abut the bone of femur 24 or a layer of bone cement may be positioned between the bone and stem 22. Articulating head 16 of femoral prosthesis may be permanently affixed to stem 22 or it may be a modular piece fit on the femoral stem at the time of surgery. After acetabular bearing 12 and/or shell 30 and femoral prosthesis 14 have been implanted, head 16 is inserted into concave bearing surface 34 of acetabular bearing to partially or completely restore normal hip joint function.

FIGS. 2A and 2B are a plan view and a section view, respectively, of an example convertible acetabular bearing in accordance with this disclosure. FIG. 2A is a plan view of acetabular bearing 12 looking down at a polar region of the hemispherical bearing. FIG. 2B is a section view of acetabular bearing 12 cut along section line A-A, which is shown in FIG. 2A. Acetabular bearing 12 may be employed in hip prostheses, including hip prosthesis 10 as illustrated in FIGS. 1A and 1B, but is illustrated in greater detail in FIGS. 2A and 2B to show the structural features on radially outward convex surface 36 of bearing 12 by which the bearing is configured to be mechanically coupled either directly to an acetabulum or to a mounting structure like an acetabular cup shell.

Referring to FIGS. 2A and 2B, acetabular bearing 12 is configured to be convertible between direct connection with the acetabulum 26 of a patient's hip, e.g. as illustrated in FIG. 1A, and connection to a mounting structure, which structure is then connected to the acetabulum, as illustrated in FIG. 1B. To enable convertible placement, acetabular bearing 12 includes a number of structural features on radially outward convex surface 36, which configure acetabular bearing 12 to alternatively connect directly to the acetabulum of a patient's hip or connect to a mounting structure, e.g., acetabular cup shell 30, which is then connected to the acetabulum. In general, convex surface 36 of acetabular bearing 12 includes at least one surface feature that configures bearing 12 to be mechanically coupled to an acetabulum and at least one surface feature that configures bearing 12 to be mechanically coupled to a mounting structure configured to be connected to an acetabulum.

In the example of FIGS. 2A and 2B, acetabular bearing 12 includes radially inward concave bearing surface 34 and a radially outward convex surface 36. Both inner and outer surfaces 34 and 36, respectively, of bearing 12 are generally hemispherical. Radially outward convex surface 36 also includes planar surface 48 arranged generally the polar region of acetabular bearing 12 indicated by polar axis 56 in FIG. 2B. Acetabular bearing 12 terminates, opposite the polar region, at an equatorial region indicated by equatorial axis 52.

Acetabular bearing 12 includes male taper 54. Male taper 54 is formed by conical surface 56, which extends (up in the view of FIG. 2B) from the equator of and along convex outer surface 36 of acetabular bearing 12. Conical surface 56 effectively removes a portion of the generally hemispherical outer surface 36 of acetabular bearing 12 to form male taper 54 in bearing 12. Male taper 54 configures acetabular bearing 12 to be mounted to a mounting structure including a complementary female taper configured to receive male taper 54 formed by conical surface 52.

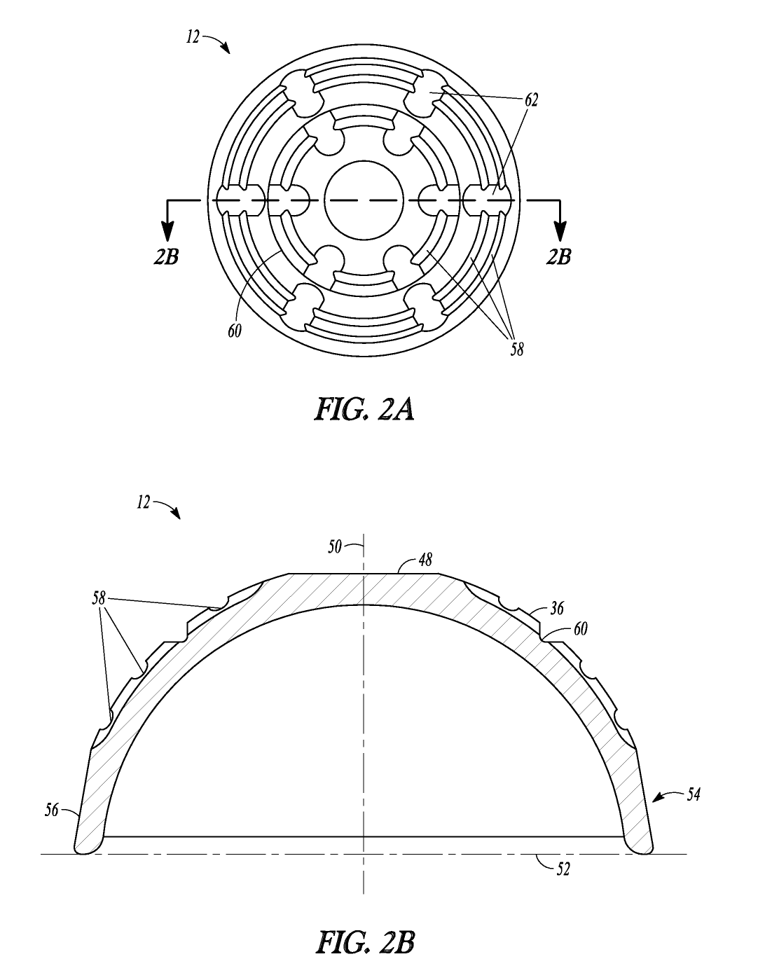

With reference to the example of FIG. 3, in which acetabular bearing 12 is coupled to cup shell 30, acetabular cup shell 30 includes a conical surface extending from the equator of and along concave inner surface 34 of shell 30 to form complementary female taper 70 configured to receive male taper 54 formed by conical surface 56 of acetabular bearing 12. In this manner, acetabular bearing 12 is configured to be mechanically coupled to a mounting structure like acetabular cup shell 30 by a taper-lock, which is also referred to as a self-locking taper. Acetabular bearing 12 interlocks with acetabular cup shell 30, which is configured to be coupled to an acetabulum, by means of male taper 54 formed on convex surface 36 mated with complementary female taper 70 formed on inner concave surface 42 of shell 30. To ensure a secure fit between acetabular bearing 12 and acetabular cup shell 30, the taper angle is chosen to be within the range of self-locking tapers. In one example, the angle, t, of male taper 54 of acetabular bearing 12 relative to female taper 70 of acetabular cup shell 30 in which male taper 54 is configured to be received is in a range from about 1 to about 35 arcminutes, or, from about 1/60 degrees to about 35/60 degrees.

In one example, a total included taper angle (both sides of the taper-lock) of an acetabular bearing and mounting structure in the range of from about 6 degrees to about 19 degrees can be employed. In some cases, to allow for removal of bearing 12 from a mounting structure like acetabular cup shell 30, the taper angle is chosen towards the high end of the range of total included taper angle for locking tapers. For example, if both acetabular bearing 12 and acetabular cup shell 30 are metallic, a total included taper angle of about 14° may be used. In order for the self-locking taper between acetabular bearing 12 and a mounting structure like acetabular cup shell 30 to function properly during implantation and use of a prosthesis, both bearing 12 and the mounting structure may need to be made of materials which will not exhibit plastic deformation, which would impair the function of the self-locking taper under the forces and at the temperatures encountered during implantation and use of the prosthesis.

In some examples, conical surface 56 that forms male taper 54 of bearing 12 may include surface features that enhance the interlock between bearing 12 and the mounting structure to which it is coupled. In one example, complementary female taper 70 of acetabular cup shell 30 may include a shallow female thread and conical surface 56 of taper 54 may be texturized such that the roughness of surface 56 is configured to engage the female thread of female taper 70 of shell 30. In one example, conical surface 56 that forms male taper 54 of bearing 12 may include a surface roughness, as specified by Rz, in a range from about 1 μm to about 30 μm.

Regardless of the particular angular relationship between or other features of male taper 54 of acetabular bearing 12 and that of the mounting structure to which bearing 12 is to be coupled, male taper 54 forms a surface feature on radially outer convex surface 36 that configures bearing 12 to be mechanically coupled to a mounting structure that can then be affixed to an acetabulum of a patient's hip. As noted above, to enable convertible placement, acetabular bearing 12 also includes at least one surface feature that configures bearing 12 to be mechanically coupled directly to an acetabulum without any intervening mounting structure like acetabular cup shell 30.

In one example, acetabular bearing 12 includes surface feature(s) on radially outward convex surface 36 that allow bearing 12 to be coupled directly to an acetabulum via an adhesive. In other words, acetabular bearing 12 can, for example, include feature(s) on surface 36 that enable or improve the ability of an adhesive (interposed between bearing 12 and the acetabulum) to bond bearing 12 to the acetabulum. In the example of FIGS. 1A-2B, acetabular bearing 12 includes a plurality of grooves in convex surface 36. The grooves in surface 36 enable interdigitation of cement mantel 38 with acetabular bearing 12 to enable or improve the ability of cement mantel 38 to bond bearing 12 to the acetabulum.

As illustrated in FIGS. 2A and 2B, acetabular bearing 12 includes multiple sets of grooves in convex surface 36 to enable/improve interdigitation of cement or other adhesive with bearing 12. Acetabular bearing 12 includes two different sets of longitudinal spherical grooves 58, 60, and a set of latitudinal grooves 62. Acetabular bearing 12 includes three longitudinal grooves 58, which are distributed between the equatorial and polar regions of bearing 12. Additionally, bearing 12 includes one longitudinal groove 60, which is interposed between two of longitudinal grooves 58. The cross-sectional profile shape of longitudinal grooves 58 is generally arcuate, while the profile shape of longitudinal groove 60 are generally triangular. Acetabular bearing 12 also includes 6 latitudinal grooves 62 that are distributed substantially equally over convex surface 36. Latitudinal grooves 62 intersect with longitudinal grooves 60 as they extend between the equatorial and polar regions of bearing 12.

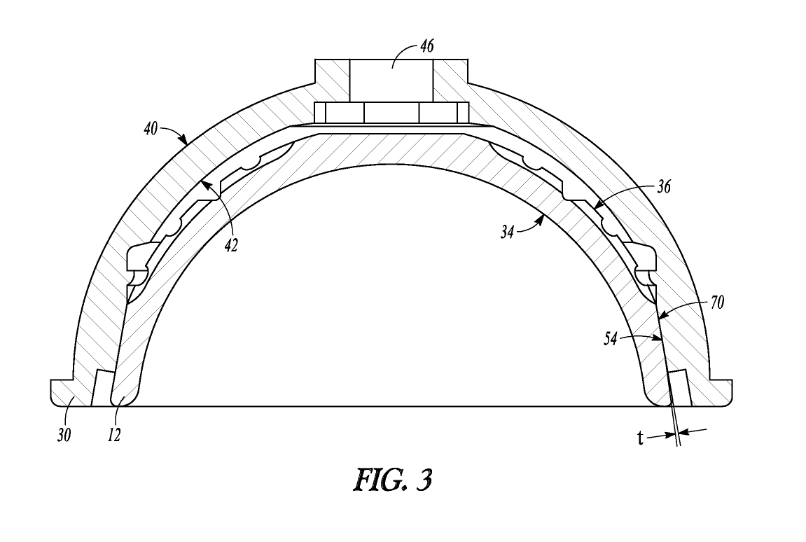

One or more of grooves 58, 60, and 62 can function to enable/improve interdigitation of cement or other adhesive with acetabular bearing 12. In another example, acetabular bearing 12 or another such bearing in accordance with this disclosure may include fewer, more, or differently configured grooves or other surface features to enable/improve mechanical coupling of bearing 12 directly to an acetabulum. For example, acetabular bearing 12 could include more than four longitudinal grooves. In another example, bearing 12 could include one helical groove that wraps multiple times around convex surface 36 between the equatorial and polar regions of bearing 12. Additionally, instead of employing elongated grooves for interdigitation, convex surface 36 may include a plurality of separate depressions distributed over the surface, e.g., surface 36 could include different numbers of dimples distributed randomly or parametrically over surface 36. Additional example variations of surface features that may be employed on an acetabular bearing to enable/improve the ability of the bearing to mechanically coupled directly to an acetabulum are illustrated in FIGS. 4A-4C.

FIGS. 4A-4C are broken section views of a portion of acetabular bearings in accordance with this disclosure. As noted above, the cross-sectional profile shape of longitudinal grooves 58 of acetabular bearing 12 in the example of FIGS. 2A and 2B is arcuate and the profile shape of longitudinal groove 60 are generally triangular. However, grooves or channels including other profile shapes may be employed. For example, acetabular bearing 100 of FIG. 4A includes a number of rectilinear shaped grooves 102 in radially outer convex surface 104.

In some examples, instead of employing grooves, channels, dimples, or other depressions in the outer surface of an acetabular bearing, other types of surface features may be employed to enable/improve the ability to couple the bearing directly to an acetabulum. FIGS. 4B and 4C illustrate two examples of acetabular bearings including different types of surface features to enable/improve the ability to couple the bearing directly to an acetabulum. In FIG. 4B, acetabular bearing 200 includes a number of protrusions 202 on radially outer convex surface 204. Protrusions 202 can be formed as discrete bosses distributed randomly or parametrically over surface 204 or as elongated ridges that extend partially or completely around surface 204.

In FIG. 4C, acetabular bearing 300 includes texturized surface 302, which includes a relatively coarse, rough surface that is configured to promote bonding of bearing 300 to an acetabulum using an adhesive. As noted, texturized surface 302 is a surface that includes a degree of roughness, which is used herein as a measure of the amount of vertical deviations of a real surface from its ideal form. Texturized surface 302 can be formed in a variety of ways including via grit blasting or etching processes. The relative roughness of texturized surface 302 can vary depending on a variety of factors, including, e.g., the material from which bearing 300 is constructed and the type of adhesive that is employed to bond bearing 300 to an acetabulum. In one example, the roughness of texturized surface 302, as defined by Rz, is in a range from about 1 μm to about 2 mm.

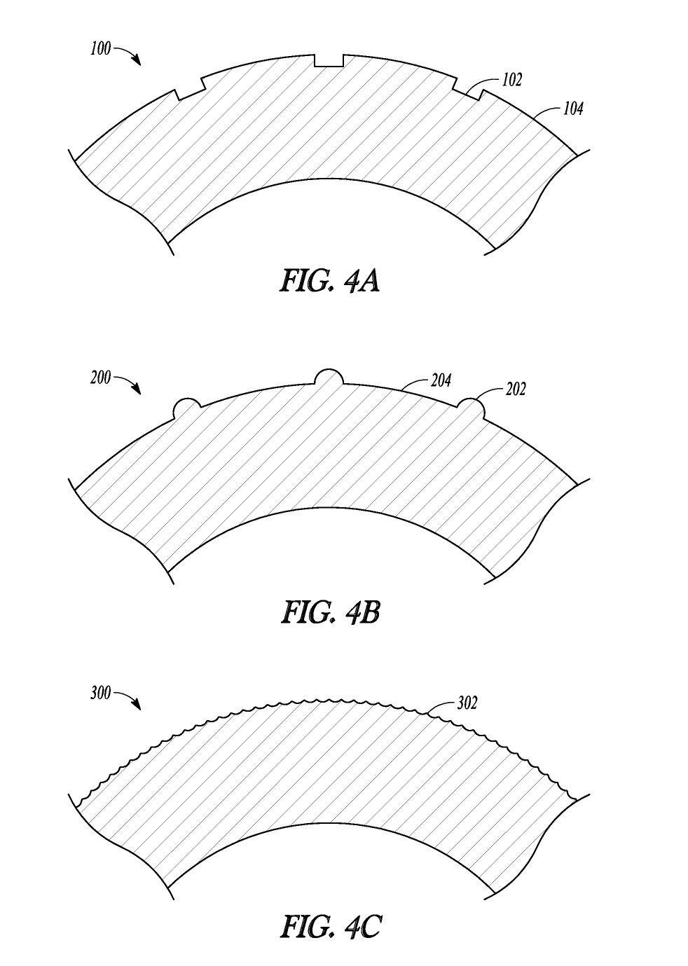

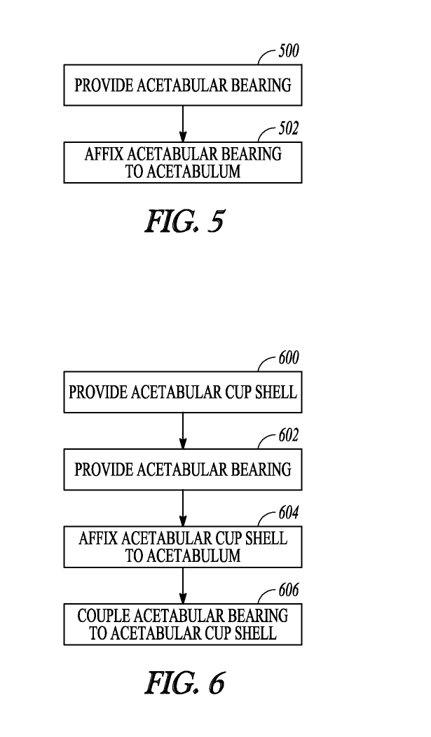

FIGS. 5 and 6 are flowcharts illustrating methods of implanting a hip prosthesis including an acetabular bearing in accordance with this disclosure. FIG. 5 is a flowchart illustrating a method of implanting a hip prosthesis in which an acetabular bearing is coupled directly to an acetabulum. FIG. 6 is a flowchart illustrating a method of implanting a hip prosthesis in which an acetabular bearing is coupled to a mounting structure that is affixed to an acetabulum.

The example method of FIG. 5 includes providing an acetabular bearing (400) and affixing the acetabular bearing to the acetabulum (402). The acetabular bearing includes a radially inner concave hemispherical surface that is configured to receive a portion of at least one of a femur or a femoral prosthesis, and a radially outer convex hemispherical surface including a first surface feature that configures the acetabular bearing to be affixed to an acetabulum and a second surface feature that configures the acetabular bearing to be coupled to a mounting structure that is configured to be affixed to the acetabulum. The acetabular bearing is affixed to the acetabulum via the first surface feature.

The example method of FIG. 6 includes providing an acetabular cup shell (500), providing an acetabular bearing (502), affixing the acetabular cup shell to the acetabulum (504), and coupling the acetabular bearing to the acetabular cup shell (506). The acetabular cup shell includes a first radially inner hemispherical concave surface, and a first radially outer convex hemispherical surface configured to be received in and affixed to an acetabulum. The acetabular bearing includes a second radially inner concave hemispherical surface that is configured to receive a portion of at least one of a femur or a femoral prosthesis, and a second radially outer convex hemispherical surface configured to be received in the first radially inner concave hemispherical surface of the acetabular cup shell. The second radially outer convex hemispherical surface includes a first surface feature that configures the acetabular bearing to be affixed to the acetabulum and a second surface feature that configures the acetabular bearing to be coupled to the acetabular cup shell. The acetabular bearing is coupled to the acetabular cup shell via the second surface feature.

In practice, an acetabular prosthesis can include just acetabular bearing 12, in accordance with example method of FIG. 5, or bearing 12 and shell 30, in accordance with the example method of FIG. 6. The acetabular prosthesis lines acetabulum 26 on the pelvic side of hip 28. Acetabular bearing 12 or acetabular bearing 12 connected to acetabular cup shell 30 is pressed into the prepared acetabulum 26 of hip 28.

In accordance with one example of the method of FIG. 5, acetabular bearing 12 is affixed to acetabulum 26 by cement mantel 38 to lock bearing 12 in place. For example, acetabular bearing 12 includes surface feature(s) on radially outward convex surface 36 that allow bearing 12 to be coupled directly to an acetabulum via an adhesive. In other words, acetabular bearing 12 can, for example, include feature(s) on surface 36 that enable or improve the ability of an adhesive (interposed between bearing 12 and the acetabulum) to bond bearing 12 to the acetabulum. In one example, acetabular bearing 12 includes a plurality of grooves in convex surface 36. The grooves in surface 36 enable interdigitation of cement mantel 38 with acetabular bearing 12 to enable or improve the ability of cement mantel 38 to bond bearing 12 to the acetabulum.

In accordance with one example of the method of FIG. 6, acetabular cup shell 30 is affixed to acetabulum 26 by cement mantel 44 to lock shell 30 in place. However, in other examples, acetabular cup shell 30 may abut acetabulum and be affixed thereto by other appropriate fixation devices, including, e.g., one or more bone screws. Additionally, in one example, in addition to cement mantel 44, acetabular cup shell 30 may also be secured to acetabulum 26 by one or more bone screws, e.g., a bone screw through hole 46 generally aligned with the pole of hemispherical shell 30.

Whether before or after affixing acetabular cup shell 30 to acetabulum 26, acetabular bearing 12 is coupled to shell 30. For example, acetabular bearing 12 can include male taper 54, which is formed by conical surface 56 extending from the equator of and along convex outer surface 36 of acetabular bearing 12. Male taper 54 configures acetabular bearing 12 to be mounted to acetabular cup shell 30 including a complementary female taper configured to receive male taper 54. For example, acetabular cup shell 30 can include a conical surface extending from the equator of and along concave inner surface 34 of shell 30 to form a complementary female taper configured to receive male taper 54 formed by conical surface 56 of acetabular bearing 12. In this manner, acetabular bearing 12 is configured to be mechanically coupled to acetabular cup shell 30 by a taper-lock, which is also referred to as a self-locking taper. To ensure a secure fit between acetabular bearing 12 and acetabular cup shell 30, the taper angle is chosen to be within the range of self-locking tapers. In one example, the angle of male taper 54 of acetabular cup 12 relative to the angle of the female taper of acetabular cup shell 30 is in a range from about 1 to about 35 arcminutes, or, from about 1/60 degrees to about 35/60 degrees.

Various examples have been described. These and other examples are within the scope of the following claims.

1. An acetabular bearing comprising:
a radially inner concave hemispherical surface that is configured to receive a portion of at least one of a femur or a femoral prosthesis; and
a radially outer convex hemispherical surface comprising at least one surface feature that configures the acetabular bearing to be affixed to an acetabulum and at least one surface feature that configures the acetabular bearing to be coupled to a mounting structure that is configured to be affixed to an acetabulum.

2. The acetabular bearing of claim 1, wherein the at least one surface feature that configures the acetabular bearing to be affixed to an acetabulum comprises one or more variations in the radially outer convex hemispherical surface that configures the acetabular bearing to be affixed to the acetabulum by interdigitation with an adhesive interposed between the radially outer convex hemispherical surface and the acetabulum.

3. The acetabular bearing of claim 2, wherein the variation in the radially outer convex surface comprises at least one depression.

4. The acetabular bearing of claim 3, wherein the at least one depression comprises at least one of an elongated groove or a dimple.

5. The acetabular bearing of claim 3, wherein the at least one depression comprises a plurality of elongated grooves.

6. The acetabular bearing of claim 5, wherein the plurality of elongated grooves comprises a plurality of longitudinal grooves and a plurality of latitudinal grooves.

7. The acetabular bearing of claim 6, wherein two or more of the plurality of longitudinal grooves comprise different cross-sectional shapes.

8. The acetabular bearing of claim 5, wherein one or more of the plurality of elongated grooves comprises a cross-sectional shape comprising at least one of an arcuate, rectilinear, or triangular shape.

9. The acetabular bearing of claim 2, wherein the variation in the radially outer convex surface comprises at least one protrusion.

10. The acetabular bearing of claim 9, wherein the at least one protrusion comprises at least one of an elongated ridge or a boss.

11. The acetabular bearing of claim 9, wherein the at least one protrusion comprises a plurality of elongated ridges.

12. The acetabular bearing of claim 2, wherein the variation in the radially outer convex hemispherical surface comprises a texturized portion of the radially outer convex hemispherical surface that comprises a surface roughness, as defined by Rz, in a range from about 1 μm to about 2 mm.

13. The acetabular bearing of claim 1, wherein the at least one surface feature that configures the acetabular bearing to be coupled to a mounting structure that is configured to be affixed to an acetabulum comprises a male taper that is configured to interlock with a female taper in the mounting structure.

14. The acetabular bearing of claim 13, wherein a relative angle between the male taper of the acetabular bearing and the female taper of the mounting structure is in a range between about 1 acrminute to about 35 arcminutes.

15. The acetabular bearing of claim 13, wherein the male taper is arranged on the radially outer convex hemispherical surface adjacent an equator of the acetabular bearing.

16. A prosthesis comprising:
an acetabular cup shell comprising:
a first radially inner hemispherical concave surface; and
a first radially outer convex hemispherical surface configured to be received in and affixed to an acetabulum; and
an acetabular bearing comprising:
a second radially inner concave hemispherical surface that is configured to receive a portion of at least one of a femur or a femoral prosthesis; and
a second radially outer convex hemispherical surface configured to be received in the first radially inner concave hemispherical surface of the acetabular cup shell, wherein the second radially outer convex hemispherical surface comprises at least one surface feature that configures the acetabular bearing to be affixed to an acetabulum and at least one surface feature that configures the acetabular bearing to be coupled to the acetabular cup shell.

17. The prosthesis of claim 16, wherein the at least one surface feature that configures the acetabular bearing to be affixed to an acetabulum comprises one or more variations in the second radially outer convex hemispherical surface that configures the acetabular bearing to be affixed to the acetabulum by interdigitation with an adhesive interposed between the second radially outer convex hemispherical surface and the acetabulum.

18. The prosthesis of claim 17, wherein the variation in the radially outer convex surface comprises a plurality of elongated grooves.

19. The prosthesis of claim 16,
wherein the first radially inner hemispherical concave surface of the acetabular cup shell comprises a female taper, and
wherein the at least one surface feature that configures the acetabular bearing to be coupled to the acetabular cup shell comprises a male taper that is configured to interlock with the female taper in the first radially inner hemispherical concave surface of the acetabular cup shell.

20. A method comprising:
providing an acetabular bearing comprising:
a radially inner concave hemispherical surface that is configured to receive a portion of at least one of a femur or a femoral prosthesis; and
a radially outer convex hemispherical surface comprising a first surface feature that configures the acetabular bearing to be affixed to an acetabulum and a second surface feature that configures the acetabular bearing to be coupled to a mounting structure that is configured to be affixed to the acetabulum; and
affixing the acetabular bearing to at least one of the acetabulum and the mounting structure via at least one of the first and second surface features, respectively.
